(19) United States
(12) Patent Application Publication
(10) Pub. No.: US 2011/0105928 A1
(43) Pub. Date: May 5, 2011

(54) ECG Reconstruction For Atrial Activity Monitoring And Detection

(75) Inventors: Bosko Bojovic, Belgrade (RS); Ljupco Hadzievski, Belgrade (RS); Dorin Panescu, San Jose, CA (US)

(73) Assignee: NewCardio, Inc., Santa Clara, CA (US)

(21) Appl. No.: 12/613,488

(22) Filed: Nov. 5, 2009

(51) Int. Cl. A61B 5/0402

(52) U.S. Cl. 600/515

(57) ABSTRACT

A system includes a mobile unit having a plurality of electrodes, numbering less than ten, that are configured to contact a patient to obtain electrical signals therefrom, and a diagnostic center disposed remotely from the mobile unit. The mobile unit and/or the diagnostic center are configured to construct a first portion of an ECG (electrocardiogram) corresponding to a first portion of a cardiac cycle of the patient by processing information based on received electrical signals using a first set of transformation parameters corresponding to the first portion of the cardiac cycle. The first portion may correspond to atrial or ventricular activity.

### CROSS-REFERENCE TO RELATED APPLICATIONS

This application may be considered related to U.S. patent application Ser. No. 10/568,868 filed Feb. 21, 2006, which claims priority to the International Application No. PCT/YU04/00020 filed Aug. 19, 2004, and U.S. patent application Ser. No. 12/534,064, filed Jul. 31, 2009.

### TECHNICAL FIELD

The present invention relates to the field of medical electronics, and more particularly to the field of instruments for measuring and recording bioelectric signals, such as electrocardiograms (ECGs).

### BACKGROUND

The concept of a system for urgent cardiac diagnostics, in which measurements for ECG (electrocardiogram) determination are obtained from the patient and sent to a remote diagnostic center for possible intervention, is known. Such systems rely on measurements taken by the patient, and then, on the basis of these and a conversation with the patient, a cardiologist at a remote location can decide: a) whether an urgent intervention is needed, b) whether the intervention can be performed by the patient himself, or c) whether the patient's state requires urgent medical intervention, and acts accordingly. There are a number of patents and products which, within the concept of urgent cardiological diagnostics, offer different solutions for recording and transmitting an ECG signal. The solutions provided by this prior art can be divided into three groups. The first group comprises solutions for sending the recording of one or two standard ECG leads. The mobile recorders of this group can be very small and with integrated electrodes (no cables are needed), which is the advantage of the group. The recording is performed by simple holding of the device on the patient's chest or by positioning the fingers on the integrated electrodes. This is a quick and simple way for a patient to record one or two leads of his ECG. However, recording one or two ECG signals limits the application of these devices to the patients with rhythm disorders, which is about 20% of the patient population with heart diseases. Moreover, because of the rudimentary nature of the analysis conducted and the small number (one or two) of electrodes used, the information that can be obtained is limited and such devices have proven to be of dubious value for the detection of atrial fibrillation, and, specifically, for discriminating atrial fibrillation from other confounding rhythms, such as atrial flutter and other supraventricular tachycardias.

The second group consists of solutions that enable direct recording and transmission of a standard 12-lead ECG, thus including their application to the patients with the diagnoses of coronary artery diseases. Namely, in such patients, the complete standard 12-lead ECG is necessary for urgent diagnostics. Some of these devices are equipped with the full set of electrodes and cables for recording all 12 standard ECG leads (usually 10 electrodes, or cables, from which 12 ECG leads are derived: 8 independent leads and 4 dependent leads). The patient in this group himself attaches the 10 electrodes onto his body during recording. The typical representative of this group is “12 Lead Memory ECG Recorder” by TELESCAN MEDICAL SYSTEMS. Another method is the use of a reduced number of electrodes that are moved during the recording. For example, if four electrodes are used, three are positioned at the locations of standard ECG leads I, II, and III (arms and legs of the patient), while the fourth electrode has to be moved during recording to each of the six chest positions for recording chest leads V1-V6 (See U.S. Pat. No. 4,889,134, Greenwold et al.). A common disadvantage of this group of devices is a rather complicated and long-lasting recording procedure, which makes them very inconvenient for self-application, especially for patients suffering a heart attack. Moreover, significant errors are possible, due to imprecise positioning of the electrodes.

The third group includes solutions in which a reduced number of special leads is recorded, and later, on the basis of this recording, all 12 standard ECG leads are reconstructed computationally. The method for the reconstruction of 12 standard ECG leads and/or x,y,z leads of a vectorcardiogram based on the recorded special leads obtained with four electrodes is disclosed in U.S. Pat. No. 4,850,370, to G. E. Dower, or instance. The method is based on the dipole approximation of the electrical heart activity and uses a universal tranformation matrix T, with dimensions 3×12, and with the matrix coefficients determined experimentally. The conventional ECG leads V (I, II, III, aVR, aVL, aVF, V₁, V₂. V₃, V₄, V₅, V₆) are obtained by multiplying the transformation matrix T with the recorded signals at the special leads Vₛ(Vₛ₁, Vₛ₂, Vₛ₃). The universal transformation matrix for all patients does not contain information about individual characteristics of a patient, which can result in major errors in the reconstruction of the standard ECG lead signals. In addition, the quality of signal reconstruction is highly dependent on the proper positioning of special leads electrodes.

An improvement to the last-mentioned approach, which uses an individual transformation matrix, is described in the paper by Scherer, J. A. et al., Journal of Electrocardiology, v 22 Suppl, pp. 128, 1989, and applied in the U.S. Pat. No. 5,058,598 (J. M. Niklas et al., 1991), wherein the implementation of the individual transformation matrix for each patient, with the segment calculation of the transformation matrix coefficients, was suggested (ECG signal is divided into segments and the coefficients for each segment are calculated individually). The reconstruction of the standard ECG lead signals by the individual transformation matrix means that it is necessary to perform the basic (calibrating) recording for each patient, which will be used for the matrix coefficient calculation. The errors in this approach are significantly reduced compared to the method using the universal transformation matrix. The major drawback of both of these methods is the need to use cables for recording with the suggested arrangement of electrodes. The method in which the reconstruction of standard ECG leads is also performed with the individual transformation matrix (Scherer, J. A. et al., Journal of Electrocardiology, v 22 Suppl, pp. 128, 1989), but with a mobile ECG device with integrated electrodes—that is, with no cables used, is described in copending U.S. Pat. application Ser. No. 10/568,868. The device enables quick and easy recording of the special ECG leads and reconstruction of all 12 standard ECG leads with the individual transformation matrix. However, this device, like all the prior art discussed above, has not been developed for monitoring and detecting atrial fibrillation and for discriminating atrial fibrillation from other, less critical rhythms, such as atrial flutter and other supraventricular tachycarias. To raise the performance of atrial fibrillation monitoring to clinically acceptable levels, the device needs to target its processing on accurate reconstruction of atrial activity, separate from the reconstruction of ventricular or isoelectric, if present, segments. None of the prior art devices provide for accurate atrial activity reconstruction based on an integrated electrode set.

### OVERVIEW

As described herein, a method includes using a mobile unit to contact a patient with a plurality of electrodes, numbering less than ten, obtaining an electrical signal from each electrode, and reconstructing a first portion of a cardiac cycle as represented by a 12-lead ECG (electrocardiogram) by combining information based on the electrical signals with a first transformation corresponding to the first portion of the cardiac cycle.

Also as described herein, a mobile unit includes a plurality of electrodes, numbering less than ten, configured to contact a patient to obtain electrical signals therefrom, and a controller configured to reconstruct a first portion of a cardiac cycle as represented by a 12-lead ECG (electrocardiogram) by combining information based on the electrical signals with a first transformation corresponding to the first portion of the cardiac cycle.

Also described herein, a system includes a mobile unit having a plurality of electrodes, numbering less than ten, that are configured to contact a patient to obtain electrical signals therefrom, and a diagnostic center disposed remotely from the mobile unit and configured to reconstruct a first portion of a cardiac cycle as represented by a 12-lead ECG (electrocardiogram) by combining information based on the electrical signals with a first transformation corresponding to the first portion of the cardiac cycle.

### BRIEF DESCRIPTION OF THE DRAWINGS

The accompanying drawings, which are incorporated into and constitute a part of this specification, illustrate one or more examples of embodiments and, together with the description of example embodiments, serve to explain the principles and implementations of the embodiments.

In the drawings:

FIG. 1 is a diagram of a system for monitoring atrial activity;

FIG. 2 is a diagram of a wearable device for use with the monitoring system of FIG. 1;

FIG. 2A is a diagram showing an electrode configuration of a belt of a wearable device;

FIG. 2B is a diagram showing a host unit-mounted electrode;

FIG. 2C is a diagram of a wearable device having a wearable component in the form of a shirt or blouse;

FIG. 2D is a schematic diagram showing a wearable device having wireless internal communication;

FIG. 2E is a block diagram showing details of an electrode transmitter module;

FIG. 2F is a block diagram showing details of an electrode receiver module;

FIG. 3 is a block diagram illustrating circuitry of a host unit that uses a common electrode;

FIG. 3A is a block diagram illustrating an alternative circuit arrangement that does not use a common electrode;

FIG. 4 is a flow diagram of a process for calibrating a bedside cardiac monitoring system and monitoring information therefrom;

FIG. 5 is an isometric view of a handheld device;

FIG. 6 is a schematic view showing the handheld device in use;

FIG. 7 is a schematic view showing placement of the handheld device on the chest of a patient; and

FIG. 8 is a schematic representation of selection of cardiac cycle segments representative of atrial, ventricular or isoelectric activity.

### DESCRIPTION OF EXAMPLE EMBODIMENTS

Example embodiments are described herein in the context of an ECG monitoring system and method for detection of fibrillation. Those of ordinary skill in the art will realize that the following description is illustrative only and is not intended to be in any way limiting. Other embodiments will readily suggest themselves to such skilled persons having the benefit of this disclosure. Reference will now be made in detail to implementations of the example embodiments as illustrated in the accompanying drawings. The same reference indicators will be used to the extent possible throughout the drawings and the following description to refer to the same or like items.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

In accordance with this disclosure, the components, process steps, and/or data structures described herein may be implemented using various types of operating systems, computing platforms, computer programs, and/or general purpose machines. In addition, those of ordinary skill in the art will recognize that devices of a less general purpose nature, such as hardwired devices, field programmable gate arrays (FPGAs), application specific integrated circuits (ASICs), or the like, may also be used without departing from the scope and spirit of the inventive concepts disclosed herein. Where a method comprising a series of process steps is implemented by a computer or a machine and those process steps can be stored as a series of instructions readable by the machine, they may be stored on a tangible medium such as a computer memory device (e.g., ROM (Read Only Memory), PROM (Programmable Read Only Memory), EEPROM (Electrically Eraseable Programmable Read Only Memory), FLASH Memory, Jump Drive, and the like), magnetic storage medium (e.g., tape, magnetic disk drive, and the like), optical storage medium (e.g., CD-ROM, DVD-ROM, paper card, paper tape and the like) and other types of program memory.

A system and method are described herein for reliably and comfortably monitoring patient ECG. Typical use is for patients who have complained of or exhibited atrial fibrillation, and who have undergone or are planning to undergo treatment for this condition. Treatment can include surgical procedures and/or medication. Recent Heart Rhythm Society Atrial Fibrillation Task Force guidelines recommend that after such treatment patients have their cardiac electrical activity monitored for up to two years. Assessment of atrial rhythm for such patients is important so that their care providers receive an accurate and well-documented understanding of the patient's atrial fibrillation burden prior to and post treatment. As described herein and pursuant to these goals, a patient can record and transmit his/her rhythm at certain times of the day, and/or when the patient becomes symptomatic.

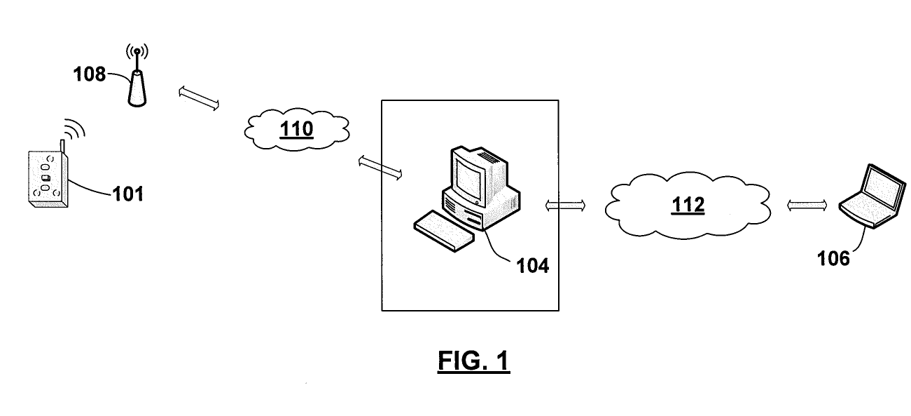

A system such as that shown in FIG. 1 can be used for the above procedure, and performs cordless/wireless recording, transmission, and processing of three special ECG leads. The measurements obtained are delivered, preferably wirelessly, to a diagnostic center where they are reconstructed, using individualized transformation matrices specific to the patient, to produce the patient's ECG in real-time for monitoring by automated equipment and/or staff at the diagnostic center or at a remote location in communication with the diagnostic center. The system, shown generally at 100, includes a mobile unit 101, which is either a wearable device 102 or a handheld device 103. The mobile unit 101 is used to extract three special ECG signals from the patient. In the case of the wearable device 102, the patient can wear it and even sleep in it comfortably for an extended period of time, such as several days, for constant monitoring. The mobile unit 101 wirelessly transmits the extracted signals, or derivation signals and information based thereon, to diagnostic center 104. Transmission can be in real time, or the information can be stored in the mobile unit 101 and transmitted in bursts at designated intervals (once each hour, etc.), or it can be retained in the mobile unit 101 for subsequent downloading at the diagnostic center 104, in the manner of a Holter-type device, for example through a USB cable or other connection, or through Bluetooth or other wireless expedient. The transmitted or stored information can be raw data, or it can be data that has been partially or completely processed at the mobile unit 101, as explained in greater detail below.

In a hospital setting in which a wearable device 102 is used, the patient (not shown) wearing the wearable device can be in one location, such as a private or shared room, while the diagnostic center 104 can be at a different location or room. The ECG generated at the diagnostic center 104 can be displayed and monitored there, or at a remote location 106 on or off the hospital premises and in communication with the diagnostic center. While a single wearable device 102 is depicted in the hospital setting, it is possible for diagnostic center 104 to be in communication with multiple such devices so that a plurality of patients can be monitored simultaneously by the single diagnostic center. Of course in that case, when individualized transformation matrices are used, the diagnostic center 104 would contain multiple individualized transformation matrices associated with each specific patient so that the individual patient's ECG can be reconstructed.

Also shown in FIG. 1 is a wireless access point 108 with which diagnostic center 104 can communicate with wearable device 102. One or more such access points may be provided to improve access, especially when multiple patients using multiple wearable devices and located at different regions in a care facility such as a hospital are involved. Communication between wearable device 102 and diagnostic center 104, and between the diagnostic center and the remote location 106, may be by way of one or more networks, such as local area network (LAN) 110, wide area networks (WANs), the Internet 112, and so forth, and paths between devices can be wireless or wired, or a combination of wireless and wired, and can pass through no networks or through one or more different networks. Those of ordinary skill in the art will recognize that many network configurations are possible to facilitate communication between wearable device 102 and diagnostic center 104, and these may be functions of the distances between the devices, the complexity of the system, the number of wearable devices 102/patients being monitored, the number of diagnostic centers 104 involved as a multiplicity of these are also contemplated, the number of remote locations 106, and so on.

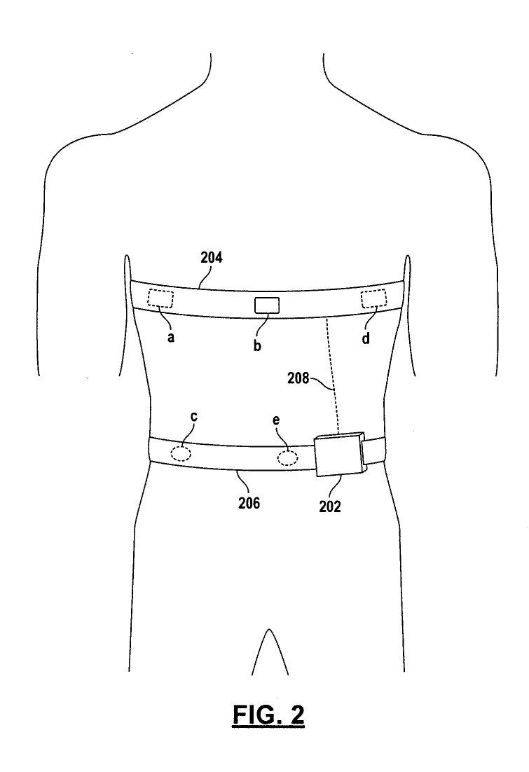

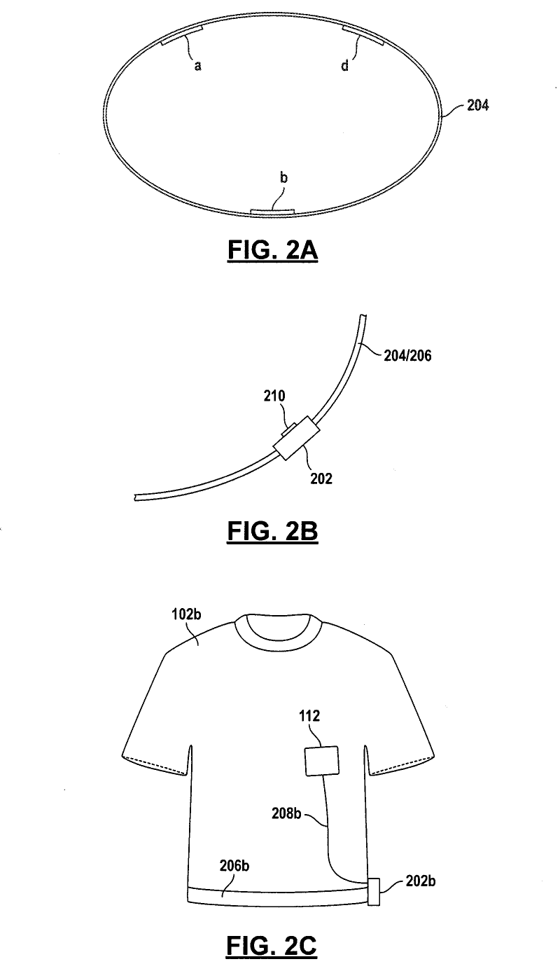

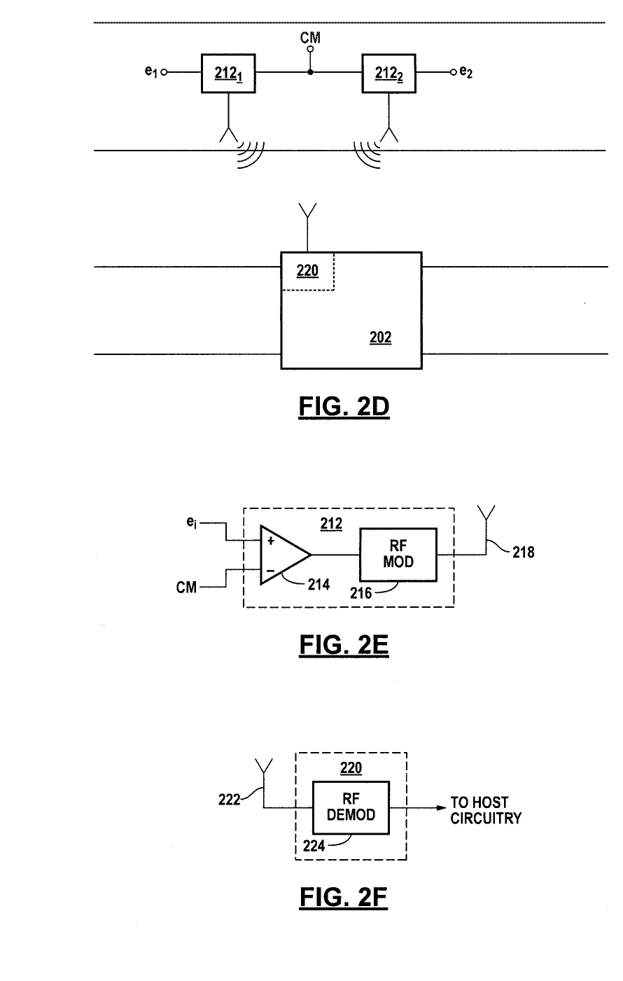

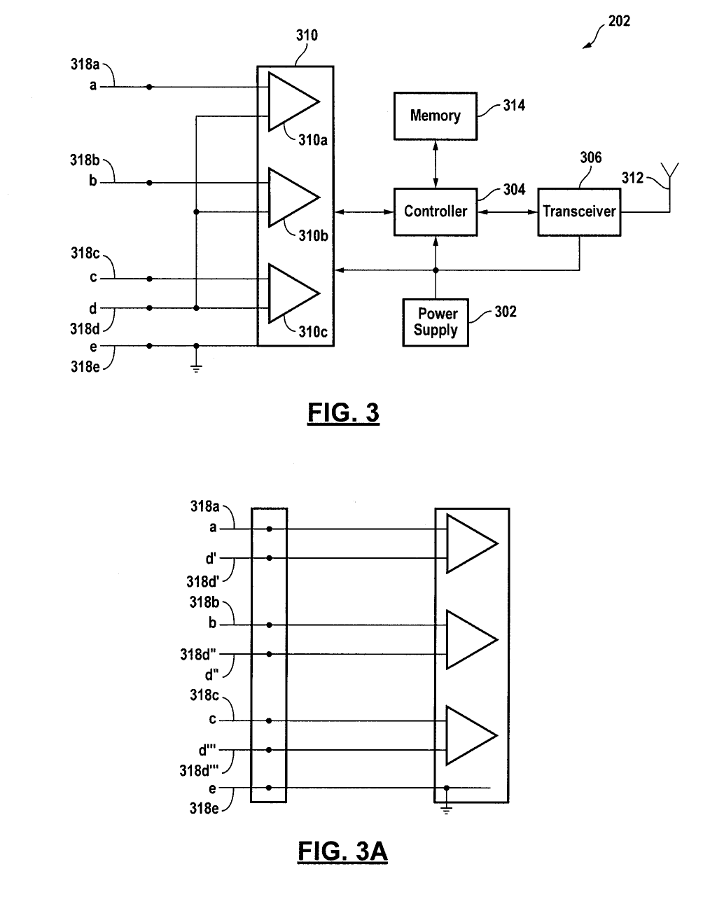

FIG. 2 shows a more detailed view of wearable device 102, which is generally in the form of a host unit 202 housing the main electrical components (detailed in FIG. 3) and coupled to wearable components in the form of a belt 204 and a waistband 206. The belt, waistband and host unit together present a set of integrated electrodes (a-e) for contact with the skin of the patient, and are distributed on the belt (belt electrodes) and waistband (waistband electrodes), and possibly the host unit (host unit electrodes), in accordance with a non-coplanar arrangement for establishing an orthogonal electrode pattern as explained below. The number of integrated electrodes in the this example is five, although this number can be greater than or less than five. In general, however, the device will likely have less than the ten electrodes used with conventional 12-lead ECG devices.

In the arrangement of FIG. 2, the host unit 202 is physically coupled to the waistband 206, but this is not mandatory and the host unit can instead be coupled to the belt 204, or can be independent of the two. In any of these cases the host unit 202 is in electrical communication with the belt electrodes and waistband electrodes such that signals from the patient acquired by these electrodes are delivered to the host unit. Depending on the exact configuration and the contemplated electrode distribution, a wired or wireless connection 208 can be provided between the host unit 202 and belt 204, and possibly an additional wired or wireless connection (not shown) can be provided between the host unit and the waistband 206.

Different schemes for the integrated electrodes (a-e) can be utilized, with the scheme shown in FIGS. 2 and 2A serving as merely one example. Importantly, an orthogonal electrode pattern is established, using at least four electrodes from which three special leads are derived. These at least four electrodes should be placed in a non-coplanar arrangement. Belt 204 is intended to be worn around the chest of the patient and as illustrated contains belt electrodes a, b, and d that contact the skin of the patient in the vicinity of his/her chest, upper waist and/or back. The electrodes a, b and d housed on waistband 206 are set about 120° apart in this example. Waistband 206 is intended to be worn around the lower waist of the patient and contains waistband electrodes c and e that contact the skin of the patient's lower waist. Other arrangements, including an opposite arrangement in which three electrodes are housed on belt 204 while two are housed waistband 206 are also contemplated. Also, while in this illustrative embodiment none of the electrodes are disposed on the host unit 202, it is possible to house one or more electrodes on the host unit 202 and mount the host unit to the waistband (or belt) such that these one or more host unit electrodes contact the skin of the patient. Such an example is shown in FIG. 2B, in which a host unit electrode 210 is shown disposed on the interior surface of host unit 202, which can be mounted on either belt 204 or waistband 206. Those of ordinary skill will recognize that other non-coplanar electrode arrangements are possible, and the invention is not limited to the specific example given herein.

It should be noted that while the wearable components of the wearable device 102 are in the form of a “belt” and a “waistband,” other expedients such as harnesses, bands, patches and straps can be used in lieu of or in conjunction with one or both the belt and waistband, and can be associated with parts of the body of the patient other than the waist and chest. For example, either or both the belt and waistband can be replaced with patches that abut the skin of the patient, bringing it into contact with electrodes disposed on the patches. Such patches can be adhered to the skin using an appropriate adhesive, or they can be sewn or otherwise affixed to the interior of a special garment worn by the patient, or they can be strapped to the patient's body through any suitable means. FIG. 2C is directed to a garment arrangement, and shows a wearable device in which the wearable component is in the form of a shirt or blouse 102b having a patch 112 housing one or more patch electrodes (not shown), a waistband 206b housing one or more waistband electrodes, and a host unit 202b communicating with the patch and waistband electrodes, through an illustrative wired connection 208b, and transmitting signals therefrom to a diagnostic center (not shown).

All the integrated electrodes (a-e) are connected to host unit 202 and provide electrical signals derived from the body of the patient to the host unit. Electrodes a-c provide special lead signals, electrode d provides a common signal, and electrode e provides a ground signal for the ECG reconstruction procedure as further explained below. The common signal from electrode d is used to efficiently provide a common reference point against which the potentials at electrodes a, b and c are measured. Alternatively, each of the electrodes a, b and c can be associated with its own reference point against which the potential is determined.

As mentioned above, some or all of the integrated electrodes a-e can communicate wirelessly with the host unit. A general schematic of such wireless communication between two electrodes in this example and the host unit is shown in FIG. 2E. The electrodes e₁ and e₂ are each shown to be associated with a dedicated electrode transmitter module 212₁ and 212₂, although it is contemplated that the transmitter modules can be shared among two or more electrodes. A common electrode CM, for providing a reference signal, is also shown, coupled to the transmitter modules 212₁ and 212₂. As discussed above, a reference electrode can be provided for each of the electrodes individually, rather than using a common electrode to provide the reference for multiple electrodes. Details of the transmitter modules 212₁ and 212₂ are shown in FIG. 2F. Specifically, the electrode signal e₁ is provided as a first input to a differential amplifier 214, and the common (or dedicated) reference electrode is provided as the second input. The output of the differential amplifier is provided to an RF (radio frequency) modulator 216 for transmission by way of an antenna 218.

The signals from transmitter modules 212₁ and 212₂ are received by a counterpart electrode receiver module 220 at the host unit 202. The receiver module 220 includes an antenna 222 and an RF demodulator 224. It is also contemplated that some or all the circuitry and components of transceiver 306, antenna 312 and controller 304, discussed in detail below, can be used to receive and process the signals from the wireless electrodes e₁ in lieu of or in addition to the circuits and components of receiver module 220.

Details of host unit 202/202b are shown schematically in FIG. 3. These include power supply 302, controller 304, wireless transceiver 306, electrode interface 308, amplifier module 310 containing amplifiers 310a-310c, antenna (internal or external) 312, and memory 314. Memory 314 can comprise a main memory and a circular buffer type memory (not shown), as discussed below. Electrode interface 308 receives electrical signals from leads 318a-318e coupled respectively to electrodes (a-e) (FIG. 2) and couples these electrically into the host unit as shown. Specifically, special leads 318a-318c are connected as inputs to corresponding amplifiers 310a-310c, common lead 318d is connected commonly to all three amplifiers 310a-310c as a reference input for special leads 318a-318c, and lead 318e is connected to ground for the three amplifiers. In the alternative embodiment mentioned above, three leads 318d′, 318d″ and 318d′″ connected respectively to electrodes d′, d″ and d′″ which are associated respectively with special electrodes a, b and c, can be used in lieu of common lead 318d. Such a configuration is illustrated in FIG. 3B. Electrode e connected to lead 318e is optional and provides improvements in noise rejection, serving to better equalize the patient potential to that of circuitry involved. As such, the minimum number of electrodes is four—a-c to provide the special leads, and d to operate as the common point of these. Electrode e is optional and serves to improve performance when needed.

The amplifiers 310a-310c amplify the signals from special leads 318a-318c and pass them to controller 304, which is optional and which can be used to provide management and control functions for the other components of the host unit 202 and wearable device 102. For instance, the operation of the amplifier module 310 can be monitored using controller 304 and feedback to the wearer or caretaker indicative of proper operation can be provided. As an example, the controller 304 can check for appropriate voltage levels received from the amplifiers, and if these are below predetermined thresholds, an indication that a lead is not properly positioned on the body of the patient can be provided, in the form of an acoustic tone or flashing LED (not shown), for instance. Conversely, proper connection and operation can be indicated by a different tone or an uninterrupted LED emission, or other indication. These indications can be provided at the wearable device 102, and/or at the diagnostic center 104 with which it is in communication. The indications can also be provided to guide the patient and/or caretaker during the calibration process detailed below, to for instance indicate successful or unsuccessful calibration, recording in-progress, and so on. The term controller, it will be appreciated, can be any type of processor that suitable for the functions ascribed herein.

Other functions of the controller 304 can be to condition the signals received from the amplifier module 310 for transmission by transceiver 306 and antenna 312. Conditioning may include appropriately modulating a carrier wave for RF transmission, in accordance with any known protocol. Other components to facilitate transmission can be used, such as a modem, as is known, and any of myriad types of wireless or wired schemes for communication between wearable device 102 and diagnostic center 104 may be employed. Moreover, two-way communication is contemplated, such that antenna 312 and transceiver 306 can be configured to receive signals from diagnostic center 104 and/or other devices to pass on to controller 304. These signals can be for performance of a handshaking procedure for proper connection, an authentication procedure, or they can be command signals for controller 304, for example to recalibrate, or to provide a failure signal or indication at the wearable device 102.

The signals from amplifier module 310 can also be stored for subsequent downloading, and memory 314 is provided for this purpose. Memory 314, which may comprise a main memory and a circular type buffer memory (not shown), is preferably a persistent type device, such that information remains stored even after power-down. Power to the various components is provided by power supply 302, which can take the form of a rechargeable or disposable battery pack.

Using the above arrangement, it is possible to precisely reconstruct all 12 signals of standard ECG leads with only signals from the three special leads 201a-201c. This is performed by combining the special leads signals with pre-generated transformation matrices, preferably ones that is individualized for the particular patient. The reconstruction is conducted either on the wearable device or at the diagnostic center, which can contain suitable computing resources that may include hardware and/or software to perform the combining, generate the ECG therefrom, display it to the operator, and possibly conduct intelligent automated monitoring and signal any alarm conditions. More details of this reconstruction and analysis, particularly with respect to cardiac activity including atrial and/or ventricular activity, is provided below.

The information obtained from integrated electrodes a-e can be transmitted to diagnostic center 204 in real time, or it can be stored, for example in memory 314, for subsequent transmission, at prescribed intervals or in a single burst. Alternatively, the information can be retained in the memory 314 (or portions thereof) for subsequent downloading at the diagnostic center, using a dedicated connection such as a cable, cradle, or wirelessly (BlueTooth™, etc.). Since the data used is from three special leads (and reference and/or ground), rather than the conventional 12 leads, the amount of information that needs to be stored is reduced and the memory requirements are similarly reduced. The information itself can be in raw form, or it can have undergone partial or complete processing in the host unit 202 to render an ECG for presentation to the caregiver or physician. Processed data is derived by combining the raw data with the personalized or general transformation matrices, for instance, which in this example would take place in the wearable device itself, and specifically in the host unit thereof. Alternatively, the 12-lead reconstruction could take place at the diagnostic center 104.

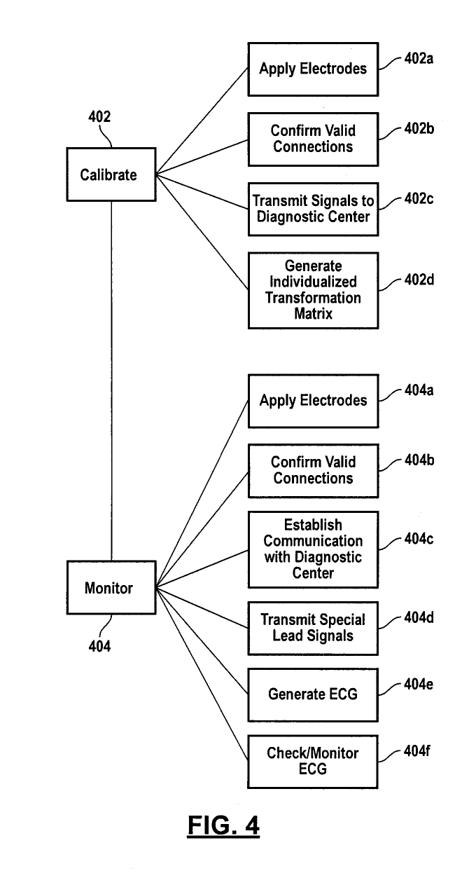

The process for monitoring a patient with the system 100 is explained with reference to FIG. 4. Initially, the individualized transformation matrices are calculated during a calibration step 402 in which a standard 12-lead ECG measurement is taken, using the wearable device 102 and a conventional ECG device having 12 actual leads, provided that this arrangement enables simultaneous recording of 3 special leads and conventional 12 leads. At 402a, the measuring device is fitted to the patient; at 402b proper connections are ascertained; at 402c signals of 3 special leads and conventional 12 leads are transmitted to diagnostic center; and at 402d the individualized transformation matrices are generated. In some situations a general transformation matrix may be used, and step 402 omitted.

Actual monitoring occurs after the calibration step, and is illustrated at 404 in FIG. 4. This includes the patient wearing wearable device 102, with belt 204 around the patient's chest and waistband 206 around the patient's lower waist, such that electrodes a-e come into contact with the patient's skin. Monitoring begins at 404a with fitting the patient with the wearable device 102. At 404b, confirmation of proper electrode-skin contact is performed. At 404c, a connection (wired or wireless) between wearable device 102 and diagnostic center 104 at 404c is established. At 404d, the three special lead signals are transmitted from wearable device 102 to the diagnostic center 104 over the established connection. At 404e, the 12-lead ECG of the patient is generated by combining the special lead signals with the individualized transformation matrix or matrices of the patient. At 404f, the ECG is checked for alarm conditions, which can be performed automatically or manually if the ECG is displayed. It will be appreciated that the step order disclosed above need not be strictly adhered to. For instance, establishment of communication between with diagnostic center 104 at step 404c can precede applying electrodes step 404a and/or confirming valid connections step 404b.

The accuracy in the reconstruction of 12 standard ECG leads using the recordings of three special leads is achieved using the arrangement of integrated electrodes as described herein. The reconstruction algorithm is based on the assumption that diffused electrical activity of the heart muscle can be approximated by a time-changing electrical dipole (heart dipole) immersed in a low conducting environment. Heart dipole is a vector defined by three non-coplanar projections, so that it can be determined on the basis of recording of electric potential in at least four points that correspond to three non-coplanar directions—that is, three ECG leads not lying on the same plane, with the fourth providing a reference, that may be common to all three (or it may be in the form of a separate electrode associated with each of the three special leads). Once the heart vector is determined, it is possible to calculate the electric potentials in any point, meaning the 12 standard ECG leads as well. The calculation of heart dipole is not necessary; the direct connection between the recorded special leads and standard ECG leads can be established instead, so that standard ECG leads are obtained as linear combinations of the recorded special leads and coefficients by which the transformation matrix is defined. However, direct application of this approach is facilitated by a detailed analysis of the error sources and an attempt to reduce them. Based on this analysis, it has been shown that there are two dominant error sources that should be taken into consideration.

### a) Model Errors

The system of reconstruction of the standard ECG leads on the basis of recording of three special leads is based on the dipole representation of heart electrical activity. However, the heart dipole is only the first term in the multipole expansion of diffused heart electrical activity and this approximation is valid only for recording points at the sufficient distance from the heart. In the points near the heart, the potential is significantly affected by the non-dipole content created due to the presence of higher order terms in multipole expansion. To reduce such errors, an assumption of at least a dual or tri-dipole model is made. Additional dipoles could be employed. One dipole is associated with the patient's atrial activity, a second dipole is associate with the patient's ventricular activity and third dipole is associated with activity that would fall in the interval that would correspond, under normal circumstance, to the isoelectric line.

### b) Transformation Matrix Calculation Errors

Practical calculation of transformation matrix T is conducted by the simultaneous recording of 12 standard ECG leads V(D₁, D₂, D₃, aVR, aVL, aVF, V₁, V₂, V₃, V₄, V₅, V₆) and three special leads Vₛ(Vₛ₁, Vₛ₂, Vₛ₃), followed by numerical solving of the equation V=T·Vₛ, by the least-squares method. The errors in recording the electric potentials introduce the errors in the calculation of transformation matrix coefficients. The analysis has shown that the errors can be minimized if the vectors of special leads recording points are orthogonal. Additionally, as described herein, the one-dipole simplification (i.e. the use of a single matrix or transformation) can introduce errors. In a dual dipole model, use of a first matrix for the atrial activity and a second matrix for the ventricular activity is made. In a tri-dipole model, use is made of a first matrix for the atrial activity, a second matrix for the ventricular activity and a third matrix, or transformation, during the period of time normally associated with the isoelectric line.

Finally, having in mind the model errors (a) and the transformation matrix calculation errors (b), two requirements are imposed concerning the arrangement of the electrodes for special leads recording, in order to minimize the total error. The first one is to position the electrodes of the special leads as far as possible from the heart; the second one is to arrange the electrodes in such a way that the vectors of recording points' positions are close to orthogonal as much as possible. By arranging the electrode positions as described herein, and choosing the common point, the optimal minimization of the model errors (a) and transformation matrix calculation errors (b) can be achieved. It should be noted that in this arrangement, the accurate positioning of the special leads is not critically important, provided that the initial positioning used in calibrations are maintained during monitoring. Furthermore, if the arrangement is to be re-applied by the patient, the application is more easy to be correctly repeated by the patient, compared to prior art.

An additional problem in signal recording of special as well as of standard ECG leads is the effect of the base line wandering of the recorded signals. The problem occurs during the recording of ECG signals with all kinds of ECG devices, but is more prominent with mobile ECG devices due to the more difficult recording conditions. When systems which obtain standard ECG leads by the reconstruction of recorded special leads are concerned, the elimination of the base line wandering problem during recording of special leads is important for the proper functioning of the system. The controller 304 can be used to establish control of the base line wandering during recording of special leads by managing the process of recording automatically. From the moment of putting the device into the recording position until the moment when the base line of a signal fits into the previously specified range, a characteristic sound signal can be emitted. During the next period defined by the signal relaxation time, another characteristic sound signal can be being emitted, indicating that the recording will start soon. The recording itself is indicated by the third characteristic sound signal. If the significant base line wandering occurs in any phase of the procedure, the procedure will be repeated from the beginning Doing so enables generation and sending of high-quality recording of special leads, which makes possible the accurate reconstruction of standard ECG leads.

The arrangement of integrated electrodes described above, their positioning, the manner of recording, and described system for eliminating the base line wandering of recorded signals minimize the errors in the reconstruction of standard ECG leads, making the accuracy of recording similar to the standard ECG devices.

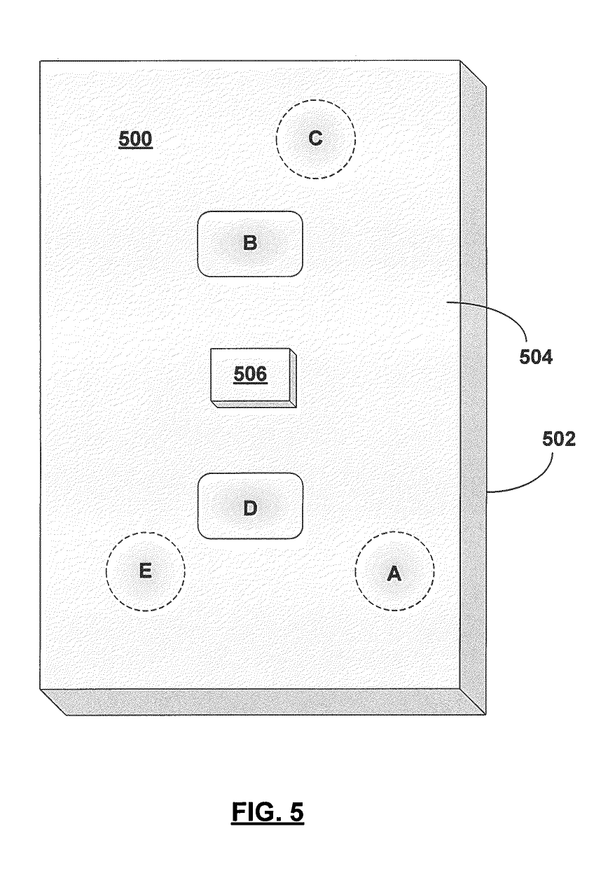

As explained above, the wearable device 102 is a specific form of a mobile unit 101 shown FIG. 1. Another specific form is a handheld device 500 shown in FIG. 5. This device is generally rectangular in shape, and has a first major surface (contact surface) 502 and a second major surface (non-contact surface) 504. The handheld device 500 includes five integrated electrodes. Two of these, B and D, are disposed on non-contact surface 504, while the remaining three (A, C and E) are disposed on the contact surface 502. In addition, one or more push-buttons 506 may be provided, serving to activate certain functions of the handheld device, such as recording and transmission of data.

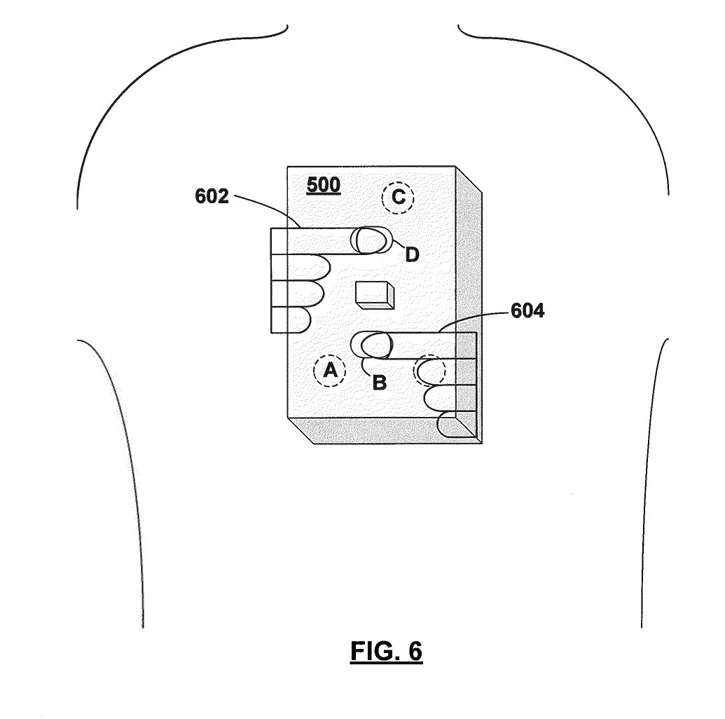

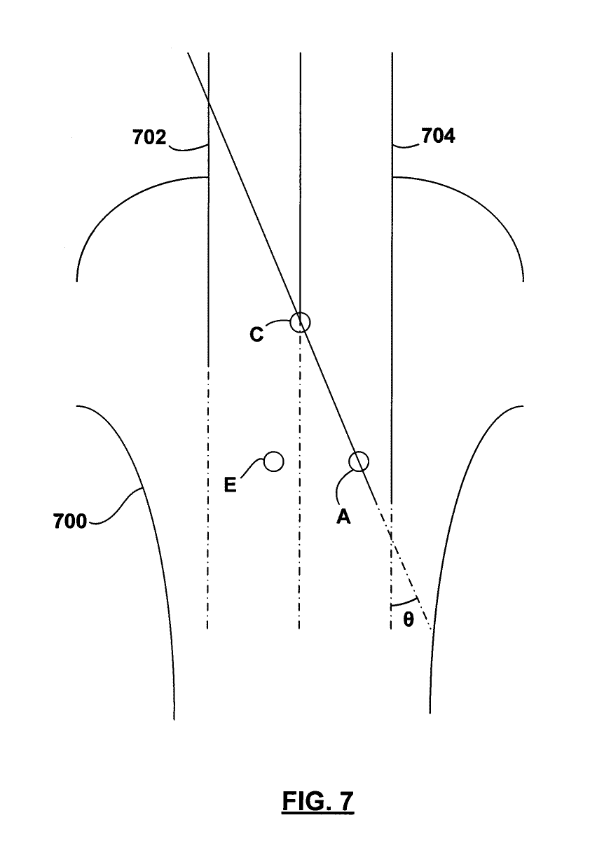

The positioning of the handheld device 500 on the chest of the patient for recording and calibration are described with respect to FIGS. 6 and 7. A patient 700 is shown in FIG. 7, with the handheld device positioned vertically in place on the patient's chest so that the electrodes A, C and E touch the patient's chest simultaneously. During recording, the device is held with a right hand finger 602 on the electrode D, and a left hand finger 604 on the electrode B (FIG. 6). The position of the active electrodes (A and C), providing the electrical contact with the patient's chest, is important for the proper functioning of the whole system. Active electrodes A and C are positioned onto the patient's chest in the area between the left (702) and the right (704) mamilar line (linea mamillaris). These electrodes should lie on the direction that, with the direction of the medial line (linea mediana anterior), makes the angle θ ranging from about 30° to about 90° as shown. The position of the electrode E, which represents the common ground, can be arbitrary, but it is convenient to choose it in such a way that it provides the mechanical stability of the handheld device while in recording position, held against the patient's chest.

The arrangement of the electrodes D and B on the non-contact surface of the handheld device 500 can be somewhat arbitrary, with primarily ergonomic considerations controlling. The position of the electrode E (common ground) can also be somewhat arbitrary, or its use can be avoided altogether if the electric scheme is solved in a different way.

Handheld device 500 contains similar electronics to host unit 202 described above, with the upper case-labeled electrodes A-E in handheld device 500 corresponding to lower case-labeled electrodes a-e in host unit 202, detailed in FIGS. 3 and 3A.

At initial set-up of the above system to monitor atrial rhythm, it is preferred that the mobile unit 101 (wearable device 102 or handheld device 500) be used simultaneously with a standard 12-lead ECG acquisition system (not shown), which for example can be a conventional 10-electrode device. Measurements are then taken from the mobile unit and the standard system and a determination of the correct patient-specific transformation parameters for conversion of the mobile unit ECG into a 12-lead ECG is made, so that proper monitoring of cardiac activity (atrial and/or ventrical) can be subsequently performed. The transformation parameter determination can be made at the diagnostic center 104, to which the measurements from the mobile unit 101 and standard ECG acquisition system are transmitted as explained above, or it can be made at the mobile unit itself Importantly, the transformation parameters for the conversion will be cardiac cycle section-specific. That is, they will be different for different sections of the cardiac cycle.

For example, but not by way of limitation, there may be a set of parameters, which can take the form of a linear or non-linear transformation or matrix and which will be referred to herein as a matrix or submatrix, for the P-Q interval (associated with atrial activity), another for Q-T (associated with ventricular activity) and yet another for the T-P_next interval (associated with what would, under normal circumstances, correspond to the isoelectric line), where P, Q, T are the well-known fiducial points of the cardiac cycle, associated with the P wave, QRS complex and the T wave. Alternatively, there may be just one set for P-Q (atrial activity) and another set for Q-P_next (ventricular activity), where P_next is the P point of the next heart beat. Yet alternatively, and in one preferred embodiment for atrial fibrillation monitoring, there is one set of conversion parameters, matrix or transformation, for Q-T (ventricular activity) and another set for T-Q_next (atrial activity), where Q_next denotes the Q point of the next heart beat. Yet another preferred atrial fibrillation monitoring embodiment uses one set of conversion parameters, matrix or transformation, from R−x to R+y (ventricular activity) and another set from R+y to R−x (atrial activity) of the next hear beat, where R is the peak of the R wave, and x and y are a preset time intervals measured milliseconds, to the left and right of the R point, respectively. For example, x could be 20 ms, whereas y could be 450 ms, thus extending passed the end of the T wave. Alternatively, x and y could be expressed as a relative amount of the current R-R interval. For example, x could be 2% of the current R-R duration, whereas y could be 45% of the current R-R duration. The reason the last two embodiments are preferred is that they do not require use of the P wave. In atrial fibrillation patients, the P wave (which represents the start of atrial activity in normal patients) is very frequently replaced by fibrillatory waves (also known as f-wave). The f-wave represents the atrial fibrillation patient's atrial activity and is, typically, asynchronous with respect to the RR interval. As such, conversion or reconstruction algorithms that make use of the P point in order to reconstruct 12-lead ECGs from a reduced set of ECGs are expected to provide decreased reconstruction accuracy during atrial fibrillatory rhythms. Since conventional reconstruction algorithms use only one single set of parameters to reconstruct the entire cardiac cycle, one advantage of the embodiments described herein is to produce an accuracy of cardiac activity reconstruction that is of clinical acceptance. This is achieved by providing patient-specific conversion parameters tailored to atrial and to ventricular activity, respectively.

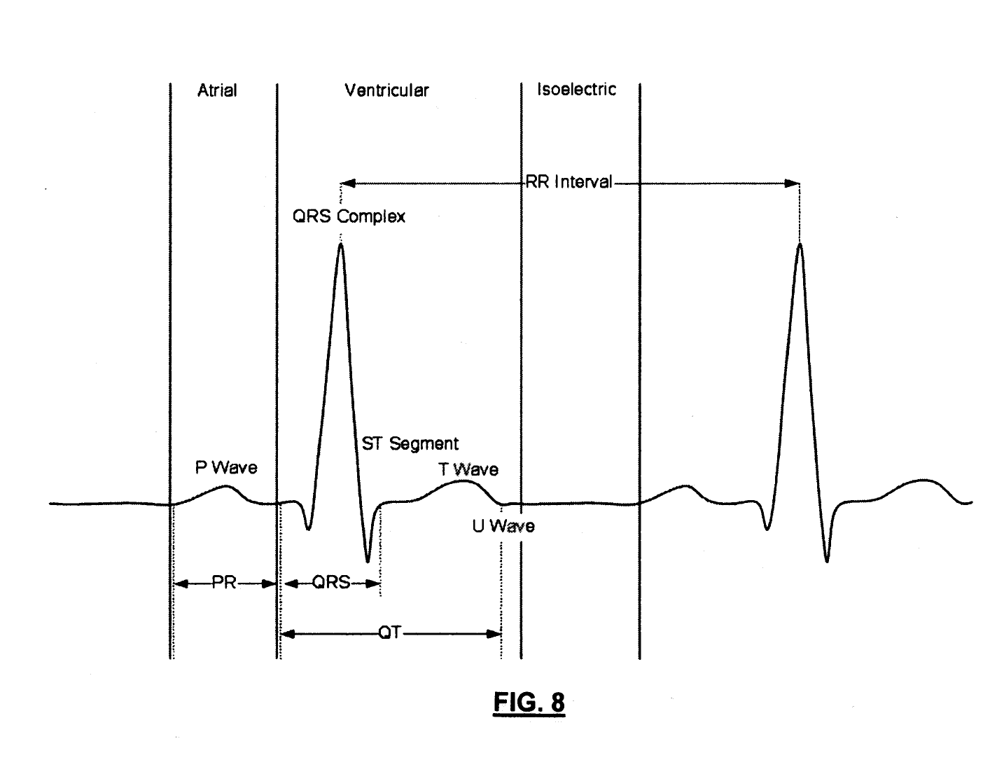

Thus, as explained above, the first step of the calibration process is to break the cardiac cycle into two or three sections corresponding to atrial activity, ventricular activity and an isoelectric line (if needed). This can be accomplished with one of the four above-identified approaches. For example, assuming that the patient is in atrial fibrillation (as such the isoelectric line might not be of relevance) the segment Q-T could represent the ventricular activity, whereas the segment T-Q_next (the Q point of the next heart beat) would represent the atrial activity. The matrix used to reconstruct the 12-lead ECG data that reflects ventricular activity is computed by matching the conventional 12-lead device data and mobile unit 101 data in the Q-T segment. This matrix is referred to as the ventricular submatrix. It corresponds to a ventricular activity portion of the cardiac cycle, which portion represents less than a complete cardiac cycle. Similarly, the matrix used to reconstruct the 12-lead ECG data that reflects atrial activity is computed by matching the conventional 12-lead device data and mobile unit 101 data in the T-Q₁₃ next (of next heart beat) segment. This matrix is referred to as the atrial submatrix. It corresponds to the atrial activity portion of the cardiac cycle, which portion also represents less than a complete cardiac cycle. A diagram of the ventricular and atrial activity portions is provided in FIG. 8. While reference is made herein to reconstruction of 12-lead ECG using matrices or submatrices, it should be recognized that such use is not intended to be limited to matrix multiplication, as other types of transformations, linear and non-linear, and involving multiplication or otherwise, are contemplated. Thus any transformation optimally tailored to the atrial, ventricular or isoelectric activity segments of the cardiac cycle could achieve clinically accepted results in atrial fibrillation monitoring.

Once the conversion parameters are obtained, monitoring of cardiac activity can take place using only mobile unit 101 (wearable device 102 or handheld device 500). The conversion parameters are stored in the system, either in the mobile unit itself, or in diagnostic center 104. These parameters are combined with measurements taken by the mobile unit 101 during operation, and monitoring of the results is conducted, either automatically or by trained personnel. Diagnostic center 104, or the mobile unit 101, can run an automatic atrial fibrillation detection algorithm, or simply provide the reconstructed 12-lead ECG for display to a cardiologist, or to other medical staff. Typical database functions can also be made available, such as sorting by patient ID, date of service, etc.

Once the coefficients of these two matrixes are optimized, the matrixes will be used to reconstruct, in real-time or off-line, the 12-lead ECG data. This can be achieved as follows:
In the incoming measurement data from the mobile unit 101, fiducial points such as P, Q, R, T, are determined. As explained above, in one example, only the Q and T points may be of interest. Algorithms for automatic detection of such fiducial points are known and described for example in U.S. Pat. No. 7,266,408, the contents of which are incorporated herein by reference in their entirety.
Once Q and T are determined, the mobile unit measurement data between Q-T are transformed to 12-lead ECG reconstructed data by using the matrix determined above for ventricular activity (the ventricular submatrix).
The mobile unit measurement data between T and next Q are converted to 12-lead ECG data by using the second matrix, which corresponds to atrial activity (atrial submatrix).
The two reconstructed 12-lead data sets are then ‘stitched’ together to form a contiguous data set. Alternatively, the two data sets can be processed prior to ‘stitching’. For example, filters could be employed to eliminate any offsets that may have been created by the use of two separate matrixes.

Once the reconstructed 12-lead ECG data set is available, processing algorithms are employed to automatically determine atrial fibrillation and to distinguish it from atrial flutter, and/or to monitor other cardiac activity. Alternatively, review by medical staff could be employed. Methods and algorithms for conducting automatic monitoring are known in the art and need not be detailed herein. The reconstructed data set can additionally or alternatively be used to display a 12-lead ECG, either graphically on paper or electronically on a monitor or the like for monitoring by a human caregiver. With reference again to FIG. 1, it will be appreciated that the reconstruction and analysis can take place either partially or fully at the mobile unit 101 (via controller 304 in FIG. 3), or at the diagnostic center 104, or at a combination of these to locations. In addition, the resultant ECG can be monitored at either of these locations (if the mobile unit 101 is suitably equipped), or it can be monitored, for example by a human caregiver, at a third location, who can access the necessary information through a website hosted at the diagnostic center 104 or by a third party at a different location.

One application of the system as described herein is as an event monitor that uses the reduced set of electrodes of the mobile unit 101. The wearable device 102 is particularly well-suited for this type of application, which would be worn for the duration of the event monitoring. The conversion/reconstruction process described above can accurately reconstruct both the atrial and the ventricular activity, as desired. In addition or alternatively, it is contemplated that automatic atrial fibrillation detection can trigger data collection. For example, recorded patient data can be stored in a circular memory buffer until atrial fibrillation (or other cardiac activity of interest) is detected. If no such activity is detected, then the controller (304, FIG. 3) in the mobile unit would cause clearing or over-writing of the data, once the length of the circular buffer is reached, or based on other factors such as cardiac cycles stored, time of day, and storage period duration. With present solid-state memory devices, buffer memory durations in excess of 10 minutes can be implemented. Alternatively, patients can press a button or the like (for example button 506 in FIG. 5) to trigger data recording when they have symptoms. If atrial fibrillation or the like is detected, the controller can cause the data corresponding to the atrial fibrillation rhythm to be stored more persistently, for example in device main memory. In addition or alternatively, an indication or notification of the atrial fibrillation or other activity of interest can be transmitted to the diagnostic center 104. The indication can be the data corresponding to the activity of interest, or the reconstructed cardiac cycles or portions thereof.

While embodiments and applications have been shown and described, it would be apparent to those skilled in the art having the benefit of this disclosure that many more modifications than mentioned above are possible without departing from the inventive concepts disclosed herein. The invention, therefore, is not to be restricted except in the spirit of the appended claims.

What is claimed is:

1. A method comprising:
using a mobile unit to contact a patient with a plurality of electrodes, numbering less than ten;
obtaining an electrical signal from each electrode; and
constructing a first portion of an ECG (electrocardiogram) corresponding to a first portion of a cardiac cycle of the patient by processing information based on the electrical signals using a first set of transformation parameters corresponding to the first portion of the cardiac cycle.

2. The method of claim 1, wherein the constructed first portion of the ECG corresponds to ventricular activity.

3. The method of claim 1, wherein the constructed first portion of the ECG corresponds to atrial activity.

4. The method of claim 1, further comprising constructing a second portion of the ECG corresponding to a second portion of the cardiac cycle of the patient by processing information based on the electrical signals using a second set of transformation parameters corresponding to the second portion of the cardiac cycle.

5. The method of claim 4, wherein the constructed first portion of the ECG corresponds to ventricular activity and the constructed second portion of the ECG corresponds to atrial activity.

6. The method of claim 1, further comprising monitoring the constructed first portion of the ECG for atrial fibrillation.

7. The method of claim 1, further comprising monitoring the constructed first portion of the ECG for atrial flutter.

8. The method of claim 6, wherein said monitoring is conducted automatically.

9. The method of claim 6, further comprising over-writing and/or erasing the constructed first portion of the ECG if no atrial fibrillation is detected.

10. The method of claim 1, wherein the plurality of electrodes is less than 6 electrodes.

11. The method of claim 1, wherein said constructing is conducted at least partially by a remotely disposed device to which information based on the electrical signals is transmitted.

12. The method of claim 1, wherein said transmission is conducted in real-time.

13. The method of claim 4, further comprising combining the constructed first and second portions of the ECG into a combined ECG.

14. The method of claim 13, further comprising monitoring the combined ECG for atrial fibrillation.

15. A mobile unit comprising:
a plurality of electrodes, numbering less than ten, configured to contact a patient to obtain electrical signals therefrom; and
a controller configured to construct a first portion of an ECG (electrocardiogram) corresponding to a first portion of a cardiac cycle of the patient by processing information based on the electrical signals using a first set of transformation parameters corresponding to the first portion of the cardiac cycle.

16. The mobile unit of claim 15, wherein the constructed first portion of the ECG corresponds to ventricular activity.

17. The mobile unit of claim 15, wherein the constructed first portion of the ECG corresponds to atrial activity.

18. The mobile unit of claim 15, wherein the controller is further configured to construct a second portion of the ECG corresponding to a second portion of the cardiac cycle of the patient by processing information based on the electrical signals using a second set of transformation parameters corresponding to the second portion of the cardiac cycle.

19. The mobile unit of claim 18, wherein the constructed first portion of the ECG corresponds to ventricular activity and the constructed second portion of the ECG corresponds to atrial activity.

20. The mobile unit of claim 15, wherein the controller is configured to monitor the constructed first portion of the ECG for atrial fibrillation.

21. The mobile unit of claim 20, wherein the controller is configured to over-write or erase the constructed first portion of the ECG if no atrial fibrillation is detected.

22. The mobile unit of claim 15, wherein the mobile unit is a wearable device.

23. The mobile unit of claim 15, wherein the mobile unit is a handheld device.

24. The mobile unit of claim 15, wherein the plurality of electrodes is less than 6 electrodes.

25. The mobile unit of claim 18, wherein the controller is further configured to combine the constructed first and second portions of the ECG to generate a combined ECG.

26. The mobile unit of claim 25, wherein the controller is further configured to monitor the combined ECG for atrial fibrillation.

27. A system comprising:
a mobile unit having a plurality of electrodes, numbering less than ten, that are configured to contact a patient to obtain electrical signals therefrom; and
a diagnostic center disposed remotely from the mobile unit and configured to construct a first portion of an ECG (electrocardiogram) corresponding to a first portion of a cardiac cycle of the patient by processing information based on the electrical signals using a first set of transformation parameters corresponding to the first portion of the cardiac cycle.

28. The system of claim 27, wherein the constructed first portion of the ECG corresponds to ventricular activity.

29. The system of claim 27, wherein the constructed first portion of the ECG corresponds to atrial activity.

30. The system of claim 27, wherein the diagnostic center is further configured to construct a second portion of the ECG corresponding to a second portion of the cardiac cycle of the patient by processing information based on the electrical signals using a second set of transformation parameters corresponding to the second portion of the cardiac cycle.

31. The system of claim 30, wherein the constructed first portion of the ECG corresponds to ventricular activity and the constructed second portion of the ECG second portion corresponds to atrial activity.

32. The system of claim 27, wherein the diagnostic center is further configured to monitor the constructed first portion of the ECG for atrial fibrillation.

33. The system of claim 27, wherein the diagnostic center is further configured to monitor the constructed first portion of the ECG for atrial flutter.

34. The system of claim 32, wherein said monitoring is conducted automatically.

35. The system of claim 27, wherein the mobile unit is a wearable device.

36. The system of claim 27, wherein the mobile unit is a handheld device.

37. The system of claim 27, wherein the plurality of electrodes is less than 6 electrodes.

38. The system of claim 27, wherein information based on the electrical signals is transmitted to the diagnostic center in real time.

39. The system of claim 30, wherein the diagnostic center is further configured to combine the constructed first and second portions of the ECG to generate a combined ECG.

40. The system of claim 39, wherein the diagnostic center is further configured to monitor the combined ECG for atrial fibrillation.

41. A system comprising:
a mobile unit including:
a plurality of electrodes, numbering less than ten, configured to contact a patient to obtain electrical signals therefrom;
a controller configured to construct a first portion of an ECG (electrocardiogram) corresponding to a first portion of a cardiac cycle of the patient by processing information based on the electrical signals using a first set of transformation parameters corresponding to the first portion of the cardiac cycle, and to monitor the constructed first portion for cardiac activity of interest; and
a first memory configured to store the constructed first portion; and
a diagnostic center in communication with the mobile unit and disposed remotely therefrom.

42. The system of claim 41, wherein the controller is further configured to erase and/or over-write the constructed first portion of the ECG from the first memory if the cardiac activity of interest is not detected.

43. The system of claim 42, wherein erasing and/or over-writing is a function of one or more of number of cardiac cycles, memory capacity, time of day, and storage period duration.

44. The system of claim 41, wherein the controller is configured to cause transmission of an indication of the presence of the cardiac activity of interest to the diagnostic center when the cardiac activity of interest is detected.

45. The system of claim 44, wherein the indication is the constructed first portion of the ECG.

46. The system of claim 41, wherein the activity of interest is ventricular activity.

47. The system of claim 41, wherein the activity of interest is atrial activity.

48. The system of claim 41, wherein the controller is further configured to construct a second portion of the ECG corresponding to a second portion of a cardiac cycle of the patient by processing information based on the electrical signals using a second set of transformation parameters corresponding to the second portion of the cardiac cycle.

49. The system of claim 41, wherein the mobile unit is a wearable device.

50. The system of claim 41, wherein the mobile unit is a handheld device.

51. The system of claim 41, wherein the plurality of electrodes is less than 6 electrodes.
